SEQUENCE LISTING (4 sequences)

SEQ ID NO: 1 - DNA, 3099 bp, Mus musculus
  CDS (1)...(3099): Evi-1
atggcgcctgacatccacgaagaacggcagcaccgctgtgaggactgtgaccagctctttgaatccaaggcagagctagccgatcaccagaagttcccatgcagcacacctcactcggccttctccatggtggaggaggacttgcaacaaaacctggagagtgagagcgatctccgagagatccatggcaaccaggactgtaaggaatgtgaccgagttttccccgatctgcaaagcttggagaagcacatgctgtcacatactgaggagagggaatacaagtgtgatcagtgtcccaaggcatttaactggaagtccaatttaattcgccaccagatgtcacatgacagtggaaagcactatgagtgtgaaaactgtgccaaggttttcacggaccctagcaaccttcagcgacacattcgatctcagcatgttggtgcccgggctcatgcttgccccgagtgtggtaaaacatttgccacttcgtcaggcctcaaacagcacaagcacatccacagcagtgtgaagccctttatctgtgaggtctgccataaatcctatactcagttttcaaacctttgtcgtcataagcgcatgcatgctgattgcagaacccaaatcaagtgcaaagactgtggacaaatgttcagcactacgtcttccttaaataaacacaggaggttttgtgagggcaagaaccattttgcggcaggtggattttttggccaaggcatttcacttcctggaaccccagctatggataaaacgtccatggttaatatgagtcatgccaacccgggcctggctgactattttggcaccaataggcatcctgctggtcttacctttccaacagctcctggattttcctttagctttcctggtctgtttccttcgggcttataccacaggcctcctttgatacccgctagtcctcctgttaaaggattatcaagtaccgaacagtcaaacaaatgtcaaagtcccctcttgacacatcctcagatactgccagctacacaggatattttgaaggcactatctaaacacccacctgtaggggacaataagccagtggaactgctgcccgagaggtcctctgaagagaggccccttgagaaaatcagtgaccagtcagagagtagtgaccttgatgatgtcagtacaccaagtggcagtgacctggaaacaacctcgggctctgatctggaaagtgaccttgaaagtgataaagagaaatgtaaagaaaatggtaaaatgttcaaagacaaagtaagccctctgcagaatctggcttcgataactaataagaaagaacacaacaatcattccgttttctcagcatctgtagaggagcaaagtgccgtgtcaggagctgtgaatgattctataaaggctattgcgtctattgctgaaaaatactttggttctacaggactggtggggctgcaagacaaaaaagttggagcgttaccttacccttccatgtttcccctcccgttttttccagcattctctcaatcaatgtacccatttcctgatagagacttgaggtcgttacctttgaaaatggagccccaatcaccaagtgaagttaagaaactgcagaagggaagctctgagtccccttttgacctcaccactaagagaaaggatgagaagcccttgacttcaggcccctcgaagccttcaggaacaccagccacaagccaagaccagcccctggatctaagtatgggcagtaggggtagagccagtgggacaaagttgactgagcctcgaaaaaaccatgtgtttggggaaaagaaaggaagcaacatggatactaggccatcttcagatggctccttgcagcatgccagacccactcccttcttcatggaccccatttatagagtagagaaaagaaagttaactgacccgcttgaagctttgaaagaaaaatacttgagaccttctccaggattcttgtttcacccgcaaatgtcagcaattgagaacatggcagaaaagctggaaagcttcagcgccctcaaacctgaggccagcgagctcctgcagtccgtgccctccatgttcagcttccgagctcctcccaacaccctgccagagaacctgctgcggaaggggaaagagcgctacacctgcaggtactgtggcaagatatttccaaggtctgcgaacctaacacggcacttgagaacccacacaggagagcaaccttacagatgcaaatactgtgatagatcattcagcatttcttccaacctgcagcgacatgtgcgcaacatccacaacaaggagaagccatttaagtgtcatttatgtgacagatgttttggtcaacaaaccaatcttgacagacacctgaagaaacatgagaacggcaacatgtctgggacggcaacgtcctcgcctcactcagagctagaaagcgcaggcgcaatcctggatgacaaagaagatgcgtactttacagagatccgcaatttcatcgggaacagcaaccatggtagccagtctcctcggaacatggaagagaggatgaatggcagtcacttcaaggataaaaaggctttggcaaccagccaaaattcagatttattggatgatgaagaagtagaagatgaggtgttgttggatgaggaggatgaagacaatgatattcctggaaagcccagaaaggagctaggggtgactcgtttagacgaggagatcccggaggatgactacgaagaagctggtgccctggagatgagctgtaaggcgtccccggtgaggtataaagaggaagactataaatctggcctttctgctctagatcacataaggcacttcacagatagcctcaaaatgagggaaatggaagagaatcaatacactgacgctgagctgtcctccattagttcttctcatgtgccagaggagcttaaacagacgttacacagaaagtccaaatcacaggcatatgctatgatgttgtcactgtctgacaaggattccctccatcccacctcccacagttcctccaacgtgtggcacagcatggcaagggctgcagcagaatccagtgccatccagtccataagccatgtatga

SEQ ID NO: 2 - protein, 1032 aa, Mus musculus
MAPDIHEERQHRCEDCDQLFESKAELADHQKFPCSTPHSAFSMVEEDLQQNLESESDLREIHGNQDCKECDRVFPDLQSLEKHMLSHTEEREYKCDQCPKAFNWKSNLIRHQMSHDSGKHYECENCAKVFTDPSNLQRHIRSQHVGARAHACPECGKTFATSSGLKQHKHIHSSVKPFICEVCHKSYTQFSNLCRHKRMHADCRTQIKCKDCGQMFSTTSSLNKHRRFCEGKNHFAAGGFFGQGISLPGTPAMDKTSMVNMSHANPGLADYFGTNRHPAGLTFPTAPGFSFSFPGLFPSGLYHRPPLIPASPPVKGLSSTEQSNKCQSPLLTHPQILPATQDILKALSKHPPVGDNKPVELLPERSSEERPLEKISDQSESSDLDDVSTPSGSDLETTSGSDLESDLESDKEKCKENGKMFKDKVSPLQNLASITNKKEHNNHSVFSASVEEQSAVSGAVNDSIKAIASIAEKYFGSTGLVGLQDKKVGALPYPSMFPLPFFPAFSQSMYPFPDRDLRSLPLKMEPQSPSEVKKLQKGSSESPFDLTTKRKDEKPLTSGPSKPSGTPATSQDQPLDLSMGSRGRASGTKLTEPRKNHVFGEKKGSNMDTRPSSDGSLQHARPTPFFMDPIYRVEKRKLTDPLEALKEKYLRPSPGFLFHPQMSAIENMAEKLESFSALKPEASELLQSVPSMFSFRAPPNTLPENLLRKGKERYTCRYCGKIFPRSANLTRHLRTHTGEQPYRCKYCDRSFSISSNLQRHVRNIHNKEKPFKCHLCDRCFGQQTNLDRHLKKHENGNMSGTATSSPHSELESAGAILDDKEDAYFTEIRNFIGNSNHGSQSPRNMEERMNGSHFKDKKALATSQNSDLLDDEEVEDEVLLDEEDEDNDIPGKPRKELGVTRLDEEIPEDDYEEAGALEMSCKASPVRYKEEDYKSGLSALDHIRHFTDSLKMREMEENQYTDAELSSISSSHVPEELKQTLHRKSKSQAYAMMLSLSDKDSLHPTSHSSSNVWHSMARAAAESSAIQSISHV

SEQ ID NO: 3 - DNA, 1313 bp, Mus musculus
  CDS (133)...(951): PU.1
ctcagggggcaggcctgagccctgcgtctgacccacgaccgtccagtcccccgacggggcacctggtcctgagggggatccgccttgatccccaccgaagcaggggatctgaccaacctggagctcagctggatgttacaggcgtgcaaaatggaagggttttccctcaccgcccctccatcggatgacttggttacttacgattcagagctataccaacgtccaatgcatgactactactccttcgtgggcagcgatggagaaagccatagcgatcactactgggatttctccgcacaccatgtccacaacaacgagtttgagaacttccctgagaaccacttcacagagctgcagagcgtgcagcccccgcagctacagcagctctatcgccacatggagctggaacagatgcacgtcctcgatactcccatggtgccaccccacaccggcctcagtcaccaggtttcctacatgccccggatgtgcttcccttatcaaaccttgtccccagcccaccagcagagctcagatgaggaggagggtgagaggcagagccctcccctggaggtgtctgatggagaagctgatggcttggagcctgggccaggtcttctgcacggggagacaggcagcaagaaaaagattcgcctgtaccagttcctgctggacctgctgcgcagcggcgacatgaaggacagcatctggtgggtggacaaggacaaaggtaccttccagttctcgtccaagcacaaggaggcgctggcgcaccgctggggcatccagaagggcaaccgcaagaagatgacctaccagaagatggcgcgcgcgctgcgcaactacggcaagacaggcgaggtgaagaaagtcaagaagaagctcacctaccagttcagcggcgaggtgctgggccgtgggggcctggccgagcggcgcctcccgccccactgatcgcccgcagagaccgccaggctcctggaccccgccggccatagcattaacccgtcgcccggcccggacacagggaggacattcccagggccgaggcaggactgggggcccggcctcgccctcccatgcccggcctggcccgccccacccgctttgcctcccaccaggactctagcccgctccaagggccgcctgggcctcggacctcaaccgagggtcagcctggcttagtggccacggtgcttccttgggagtctggcgctggcacctttttgtatattgaatgctttttaaaaagctcttcctccccatcccctcattaaccactaaagacaagtaaaattattgacagctattctccc

SEQ ID NO: 4 - protein, 272 aa, Mus musculus
MLQACKMEGFSLTAPPSDDLVTYDSELYQRPMHDYYSFVGSDGESHSDHYWDFSAHHVHNNEFENFPENHFTELQSVQPPQLQQLYRHMELEQMHVLDTPMVPPHTGLSHQVSYMPRMCFPYQTLSPAHQQSSDEEEGERQSPPLEVSDGEADGLEPGPGLLHGETGSKKKIRLYQFLLDLLRSGDMKDSIWWVDKDKGTFQFSSKHKEALAHRWGIQKGNRKKMTYQKMARALRNYGKTGEVKKVKKKLTYQFSGEVLGRGGLAERRLPPH

(19) United States
(12) Patent Application Publication
(10) Pub. No.: US 2007/0059830 A1
(43) Pub. Date: Mar. 15, 2007

(54) Method for creating myeloid cell lines

(76) Inventors: Bruce C. Trapnell, Hamilton, OH (US); Pierre-Yves Berclaz, Carmel, IN (US)

Correspondence Address: TAFT, STETTINIUS & HOLLISTER LLP, SUITE 1800, 425 WALNUT STREET, CINCINNATI, OH, 45202-3957, US

(21) Appl. No.: 11/509,314

(22) Filed: Aug. 24, 2006

Related U.S. Application Data
(63) Continuation of application No. PCT/US05/05789, filed on Feb. 24, 2005.
(60) Provisional application No. 60/547,658, filed on Feb. 25, 2004.
(60) Provisional application No. 60/548,042, filed on Feb. 26, 2004.

(51) Int. Cl. C12N 5/06; C12N 5/08

(52) U.S. Cl. 435/455; 435/325; 435/354; 435/362; 435/366; 435/350; 435/353

(57) ABSTRACT

The methods and compositions of the present invention find use in modulating myeloid cell development, particularly differentiation and proliferation. The compositions of the invention include isolated transgenic cells, transgenic tissue, transgenic animals, and transgenic mice. The methods allow generation of myeloid cell lines suitable for tissue culture. Further the invention provides methods of modulating immune disorders, particularly myeloid disorders, more particularly leukemias.

### CROSS-REFERENCE TO RELATED APPLICATIONS

This application is a continuation of and claims priority to and benefit of PCT US05/05789, filed on Feb. 24, 2005, U. S. Provisional Application 60/547,658, filed on Feb. 25, 2004 and U.S. Provisional Application 60/548,042, filed on Feb. 26, 2004, which are herein incorporated by reference in their entirety.

### GOVERNMENT GRANT INFORMATION

This invention was made with Government support under N.I.H. Grant Nos. HL69549 and HL71823. The United States Government has certain rights in this invention.

### FIELD OF THE INVENTION

The invention relates to the field of myelopoiesis, modulation of myelopoiesis, and treatment of myelopoietic disorders such as leukemias.

### BACKGROUND OF THE INVENTION

Hematopoiesis is a process in which a self-renewing, pluripotent stem cell gives rise through a series of cell divisions to the eight major cell types of the blood. During its differentiation the pluripotent stem cell appears to generate a hierarchical array of developmental intermediates, consisting of multipotent and lineage-committed progenitors. The latter cells give rise to erythrocytes, megakaryocytes, mast cells, granulocytes (e.g. neutrophils), macrophages, and B and T lymphocytes. Progression through the hematopoietic developmental cascade involves tightly controlled patterns of gene expression that are orchestrated by a complex set of transcription factors. Mutations in genes encoding these transcription factors (e.g. GATA-1, c-myb, PU.1, Ikaros, E2A, EBF, and NF-E2) cause either multilineage or single lineage developmental defects in the hematopoietic system (DeKoter et al. (1998) EMBO 17:4456-4468, herein incorporated by reference in its entirety).

The transcription factor PU.1 is a tissue preferred ets-family member that is expressed in various lineages of the hematopoietic system (Klemsz et al. (1990) Cell 60:113-124 and Hromas et al. (1993) Blood 82:2998-3004, herein incorporated by reference in their entirety). PU.1 is encoded by the proto-oncogene Spi-1, and unregulated expression of Spi-1 leads to erythroleukemias in mice (Moreau-Gachelin et al. (1988) Nature 331:277-280). PU.1 functions in granulocytes, macrophages, and B lymphocytes. Further PU. 1 is required for the development of both myeloid and lymphoid lineages (Scott et al. (1994) Science 265:1573-1577; McKercher et al. (1996) EMBO J 15:5647-5658). Reintroduction of PU.1 via retroviral transduction restores differentiation of PU.1 −/− myeloid progenitors into neutrophils and macrophages. (DeKoter et al. (1998) EMBO 17:4456-4468, herein incorporated by reference in its entirety).

Evi-1 is a zinc-finger transcriptional regulator that promotes myeloid cell proliferation (Mucenski et al. (1988) Mol. Cell Biol. 8:301-308; Perkins et al. (1991) Mol. Cell Biol. 11:2665-1674; Morishita et al. (1992) Mol. Cell Biol. 12:183- 189; Morishita et al. (1992) Proc. Natl. Acad. Sci. 89:3937-3941; Bartholemew et al. (1997) Oncogene 14:569-577; Kurokawa et al. (2000) EMBOJ 19:2958-2968; herein incorporated by reference in their entirety). An immature myeloid phenotype is associated with Evi-1 expression in myeloid leukemic cells in acute myeloid leukemia, the blast crisis of chronic myelogenous leukemia, and myelodysplastic syndromes (Cuenco & Ren (2001) Oncogene 20:8236-8248; Mitani et al. (1994) EMBOJ 13:504-510; and Dreyfus et al. (1995) Leukemia 9:203-205; herein incorporated by reference in their entirety).

Acute myeloid leukemia is the most common leukemia in adults, the second most common leukemia in children and has a significantly worse prognosis than other leukemias. Cytologically, acute myeloid leukemia is a heterogeneous group with leukemic cells having granulocytic or monocytic features. Thus, understanding myelopoiesis and developing methods of modulating myelopoiesis are of importance. It is of particular importance to develop methods of modulating immune disorders. Development of myeloid cell lines capable of differentiation or proliferation is of importance in increasing knowledge of myelopoiesis, developing methods of modulating myelopoiesis, and modulating immune disorders, particularly myeloid disorders, more particularly leukemias.

### SUMMARY OF THE INVENTION

Compositions and methods for creating myeloid cell lines and models of immune disorders, particularly myeloid disorders are provided. The invention is based, in part, upon the observation that Evi1 and PU.1 constitute a molecular switch. The interplay between Evi1 and PU.1 regulates the transition from the anchorage-independent proliferation of undifferentiated macrophage lineage cells to terminally differentiated macrophages with a reduced rate of proliferation. The proliferative capacity of mononuclear phagocytes diminishes inversely with their maturation into terminally differentiated macrophages. Evi1 is a zinc-finger protein that promotes myeloid cell proliferation. Additionally, Evi1 is implicated in acute myelogenous leukemia. PU.1 is an ETs-family transcription factor that is critical for myelopoiesis. PU.1 is a key regulator of macrophage terminal differentiation. Modulating the Evi1/PU.1 molecular switch regulates the transition between proliferation and differentiation of macrophage cells.

Compositions of the invention include myeloid cells comprising a first expression cassette comprising a first promoter operably linked to a first nucleotide sequence of interest. The first nucleotide sequence encodes Evi1 or a fragment or variant thereof. Myeloid cells are pluripotent cells further described elsewhere herein from which are derived numerous cell types including, but not limited to, erythroid cells, granulocytes, leukocytes, monocytes, macrophages, and platelets. The Evi1 nucleotide sequence is set forth in SEQ ID NO:1; the amino acid sequence of the Evi1 polypeptide is set forth in SEQ ID NO:2. The Evi1 polypeptide exhibits proliferating activity. In an embodiment, the first promoter is operably linked to the nucleotide sequence set forth in SEQ ID NO:1. In another embodiment, the first promoter is operably linked to a nucleotide sequence having at least 90% identity to the sequence set forth in SEQ ID NO:1 and encoding a polypeptide having proliferating activity or the ability to stimulate a pathway capable of stimulating myeloid cell proliferation. In yet another embodiment, the first promoter is operably linked to a nucleotide sequence encoding a polypeptide having the amino acid sequence set forth in SEQ ID NO:2. In still yet another embodiment the first promoter is operably linked to a nucleotide sequence that encodes a polypeptide having at least 90% identity to the amino acid sequence set forth in SEQ ID NO:2 and with proliferating activity. In an embodiment the myeloid cell of the invention is from a mammal, particularly from a human, monkey, chimpanzee, rat, hamster, mouse, rabbit, rat, hamster, dog, pig, goat, or cow. In an embodiment the first promoter is constitutive. In an embodiment the first promoter is inducible.

In an embodiment, a myeloid cell of the invention further comprises a second expression cassette. The second expression cassette comprises a second promoter operably linked to a second nucleotide sequence of interest, PU.1 or a fragment or variant thereof. The PU.1 nucleotide sequence is set forth in SEQ ID NO:3; the amino acid sequence of the PU.1 polypeptide is set forth in SEQ ID NO:4. The PU.1 polypeptide exhibits differentiating activity. In an embodiment, the second promoter is operably linked to the nucleotide sequence set forth in SEQ ID NO:3. In an embodiment, the second promoter is operably linked to a nucleotide sequence having at least 90% identity to the sequence set forth in SEQ ID NO:3 and encoding a polypeptide having differentiating activity. In an embodiment, the second promoter is operably linked to a nucleotide sequence encoding a polypeptide having the amino acid sequence set forth in SEQ ID NO:4. In an embodiment the second promoter is operably linked to a nucleotide sequence that encodes a polypeptide having at least 90% identity to the amino acid sequence set forth in SEQ ID NO:4 and with differentiating activity.

Processes for modulating Evi1 levels in a cell are provided. In the process at least one myeloid cell is isolated. Expression cassettes comprising a first promoter operably linked to an Evi1 nucleotide sequence are stably transformed into the isolated myeloid cell or cells. In an aspect of the invention, the first promoter is an inducible promoter. In another aspect of the invention, the first promoter is constitutive. In an aspect of the invention, the process further comprises transforming the myeloid cells with a second expression cassette. The second expression cassette comprises a second promoter operably linked to a PU.1 nucleotide sequence. In an embodiment the second promoter is constitutive. In an embodiment the second promoter is inducible. In an aspect of the invention, the first and second expression cassettes are in one vector. In yet another aspect of the invention, the first and second expression cassettes are in multiple vectors.

Processes for modulating differentiation of a myeloid cell are provided. In the process at least one myeloid cell of the invention is provided. The process of the invention comprises monitoring differentiation of the cell. In an embodiment the cell is transformed with a second expression cassette comprising a second promoter operably linked to a second nucleotide sequence of interest. The second nucleotide sequence of interest is a PU.1 nucleotide sequence. The processes modulate the differentiation of a myeloid cell.

Methods for preparing myeloid cell lines suitable for tissue culture are provided. The methods involve providing a myeloid cell of the invention and selecting cells comprising the expression cassette. In another embodiment the cells are transformed with a second expression cassette, and cells comprising the second expression cassette are selected. The second expression cassette comprises a second promoter operably linked to a PU.1 nucleotide sequence. In an embodiment of the method, clonal populations are obtained.

Processes of modulating differentiation of a myeloid cell line are provided. The process comprises providing a myeloid cell of the invention and monitoring differentiation of the transformed cells. In another embodiment, the process involves transforming the cells with a second expression cassette. The second expression cassette comprises a promoter operably linked to a PU.1 nucleotide sequence.

Methods of assaying the differentiating activity of a compound of interest are provided. In the methods, at least one myeloid cell of the invention. The cell is incubated with a compound of interest and differentiation of the cell is monitored. In various embodiments the cell differentiates into a macrophage cell type cell, a neutrophil, or a mast cell.

Methods of assaying the capability of a compound to modulate an immune disorder, particularly a myeloid disorder, more particularly a leukemia, are provided. In the method of the invention, at least one myeloid cell of the invention is provided. The cell is incubated with a compound of interest. The invention comprises monitoring a phenotype of said cell. Phenotypes of interest include, but are not limited to, cellular maturity, cell growth, and cellular differentiation. In an embodiment the compound of interest increases cellular maturity. In another embodiment the compound of interest decreases the phenotype. In various embodiments the immune disorder is acute myeloid leukemia, chronic myelogenous leukemia, chronic granulomatous disease, or an erythroid disorder.

Methods of assaying the capability of a compound to treat an immune disorder are provided. In the method of the invention, at least one myeloid cell of the invention is provided. The cell is incubated with the compound of interest and an immune disorder phenotype is monitored. Immune disorders of interest include, but are not limited to, myeloid disorders, erythroid disorders, leukemias, acute myeloid leukemia, chronic myelogenous leukemia, and chronic granulomatous disease.

Methods for identifying anti-leukemia agents are provided. In the method of the invention, at least one myeloid cell comprising an expression cassette comprising a promoter operably linked to an Evi1 nucleotide sequence is provided. The cell is incubated with a compound of interest and a leukemia associated phenotype is monitored.

The invention provides methods of modulating the transition from the proliferation of undifferentiated macrophage lineage cells to differentiated macrophages. The methods comprise the steps of isolating a macrophage lineage cell, transforming the cell with a first expression cassette comprising a promoter operably linked to an Evi1 nucleotide sequence, and monitoring differentiation of the cell. In an aspect of the invention, proliferation is anchorage-independent. In another aspect of the invention, proliferation is anchorage-dependent. In an aspect, the method further comprises transforming the cell with a second expression cassette comprising a promoter operably linked to a PU.1 nucleotide sequence. In an aspect of the methods, a differentiated macrophage has a reduced rate of proliferation. In another aspect of the methods, a differentiated macrophage is terminally differentiated. In yet another aspect of the methods, a differentiated macrophage is intermediately differentiated.

### BRIEF DESCRIPTION OF THE DRAWINGS

FIG. 1 presents the results of Evi-1 and PU.1 expression analysis in murine mononuclear phagocytes. Panel A presents the results of RT-PCR performed on total RNA obtained from mononuclear phagocytes (mAM, lanes 1-3) and macrophages (MH-S, lanes 4 and 5). Cells used to obtain RNA for lanes 2 and 3 were transduced with PU.1 retrovirus. Cells used to obtain RNA for lanes 3 and 5 were transduced with an Evi1 retrovirus. The RT-PCR reactions contained primers specific for endogenous PU.1, Evi-l (endogenous and/or retroviral), or GAPDH. Panel B presents the results of Western blot analysis of PU.1 expression in the indicated cell lines (as in Panel A). β-actin expression levels were also assayed. Panel C presents the results of promoter activity assays performed in mAM cells, mAM cells transduced with a PU.1 expression vector, or MH-S cells transformed with a vector comprising the Evi1 promoter operably linked to a luciferase reporter. Relative luciferase light units are indicated.

FIG. 2 presents results obtained from a series of experiments performed on mononuclear phagocytes (mAM), mononuclear phagocytes expressing retroviral PU.1 (mAM+PU.1), and mononuclear phagocytes expressing retroviral PU.1 and Evi1 (mAM+PU.1+Evi-1). Panel A presents the results obtained from a [³H] thymidine uptake assay. The results are presented as cpm×4,000. Panel B presents the percentage of cells exhibiting apoptotic markers in each cell line. Panel C presents the number of adherent cells in a 10× field for each cell line.

FIG. 3 presents results obtained from a series of experiments performed on mononuclear phagocytes (mAM), mononuclear phagocytes expressing retroviral PU.1 (mAM+PU.1), and mononuclear phagocytes expressing retroviral PU.1 and Evi-1 (mAM+PU.1+Evi-1). Panel A presents the results of RT-PCR on total RNA obtained from the indicated cell lines and performed with primers specific for the indicated gene. Genes assayed by RT-PCR are F4/80, FcγRIA, FcγRIIB, FcγRIIIA, Fcr-γ, and GAPDH. Panel B presents the results of flow cytometry analysis of FcγRII/Ills expression in the indicated cell lines. The mean fluorescence is indicated. Panel C presents the phagocytic index (PI) of the indicated cell lines as determined by flow cytometry assessment of internalization of fluorescent latex beads. Panel D presents the phagocytic index of the indicated cell lines as determined by flow cytometry assessment of internalization of IgG-opsonized beads.

FIG. 4 presents results obtained from a [³H] thymidine uptake assay on the indicated cell lines. The K562 cell line is human erythroid leukemic cell line. The K562 cell line was transduced with a PU.1 retrovirus at low and high levels, as indicated.

### DETAILED DESCRIPTION OF THE INVENTION

The invention relates to compositions and methods drawn to the murine Evi1 sequence, myeloid cells comprising a transgenic Evi1 sequence, and methods of modulating Evi1 expression. Further the invention provides methods of modulating myeloid cell differentiation, methods of modulating myeloid cell proliferation, and methods of preparing an immortalized myeloid cell line. Additionally the invention provides methods of modulating immune disorders such as myeloid disorders, particularly leukemias. The invention provides methods of treating leukemias. In addition, the invention provides methods of identifying Evi1 modulating agents, myeloid cell differentiation modulating agents, myeloid cell disorder modulating agents, immune disorder modulating agents, and anti-leukemic agents.

An embodiment of the invention provides a myeloid cell stably transformed with an expression cassette comprising a promoter operably linked to a nucleotide sequence of interest such as Evi1 or fragments and variants thereof. As used herein a myeloid cell is a pluripotent stem cell of the hematopoietic system. Myeloid cells are progenitor cells for erythrocytes, leukocytes, granulocytes, basophils, eosinophils, neutrophils, monocytes, lymphocytes, macrophages, or megakaryocytes. Myeloid cells used in the invention may be intermediately differentiated and thus be capable of differentiating into a subset of the cell types described above. Further a myeloid cell used in the invention is any cell with a myeloid cell ancestor. Myeloid cells of particular interest in the invention are obtained from a mammal. Suitable mammals include, but are not limited to, humans, monkeys, chimpanzees, primates, mice, rabbits, rats, hamsters, dogs, pigs, goats, cows, sheep, goats, and guinea pigs.

Methods of isolating a myeloid cell are known in the art and described in DeKoter et al. (1998) EMBO J 17:4456-4468, herein incorporated by reference in its entirety. It is envisioned that any means known in the art of isolating a myeloid cell is encompassed by the invention. The invention encompasses isolated or substantially purified myeloid cell compositions. An “isolated” or substantially “purified” myeloid cell or myeloid cells is or are substantially free of other cell types and can be separated by from culture medium by centrifugation and washing. A substantially purified population of myeloid cells may include intermediately differentiated myeloid cells or differentiated myeloid cells. A substantially purified preparation of cells, e.g. myeloid cells, as used herein, means a preparation of cells in which at least 50% of the cells, preferably at least 70% of the cells, more preferably at least 75% of the cells, yet more preferably 80%, most preferably at least 90%, 95%, 99% or more of the cells are the subject cell, e.g., are myeloid cells.

An embodiment of the invention is a myeloid cell comprising an expression cassette comprising a promoter operably linked to a nucleotide sequence of interest. Nucleotide sequences of interest include Evi1 (SEQ ID NO:1), PU.1 (SEQ ID NO:3), and fragments and variants thereof.

Fragments and variants of the nucleotide sequences of interest and polypeptides encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence exhibit a proliferating activity for Evi1 or a differentiating activity for PU.1. Alternatively, fragments of a nucleotide sequence that are useful as hybridization probes generally do not retain biological activity. Thus, fragments of a nucleotide sequence may range from at least about 20, 25, 30, 35, 40, 45, 50, 55, 60, 65, 70, 75, 80, 85, 90, 95, 100, 150, 200, 250,300,350, 400, 450, 500, 550,600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, up to 3099 nucleotides for SEQ ID NO:1 and from at least about 20, 25, 30, 35, 40, 45, 50, 55, 60, 65, 70, 75, 80, 85, 90, 95, 100, 150, 200, 250,300, 350, 400, 450, 500, 550,600, 650, 700, 750, 800, 850, 900, 950, 1000, 1050, 1100, up to about 1313 nucleotides for SEQ ID NO:3.

A fragment of a nucleotide sequence of interest that encodes a biologically active portion of a polypeptide of interest will encode at least 15, 25, 30, 40, 50, 60, 70, 80, 90, 100, 110, 120, 130, 140, 150, 160, 170, 180, 190, 200, 210, 220, 230, 240, 250, 260, 270, 280, 290,300,310, 320,330,340, 350, 360, 370, 380, 390, 400, 410, 420, 430, 440, 450, 460, 470, 480, 490, 500, 510, 520, 530, 540, 550, 560, 570, 580, 590, 600, 610, 620,630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1033 contiguous amino acids, or up to the total number of amino acids present in the full-length Evi1 protein or 15, 25, 30, 40, 50, 60, 70, 80, 90, 100, 110, 120, 130, 140, 150, 160, 170, 180, 190, 200, 210, 220, 230, 240, 250, 260, 270, 272 contiguous amino acids, or up to the total number of amino acids present in the full-length PU.1 protein. Fragments of a nucleotide sequence or interest that are useful as hybridization probes or PCR primers generally need not encode a biologically active portion of a protein.

Thus, a fragment of a nucleotide sequence of interest may encode a biologically active portion of a nucleotide sequence of interest or it may be a fragment that can be used as a hybridization probe or PCR primer using methods disclosed below. A biologically active portion of a nucleotide sequence of interest can be prepared by isolating a portion of one of the nucleotide sequences of interest, expressing the encoded portion of the protein of interest (e.g., by recombinant expression in vitro), and assessing the activity of the encoded portion of the polypeptide. Nucleic acid molecules that are fragments of a nucleotide sequence of interest comprise at least about 20, 25, 30, 35, 40, 45, 50, 55, 60, 65, 70, 75, 80, 85, 90, 95, 100, 150, 200, 250, 300,350, 400, 450, 500, 550, 600, 650, 700, 750, 800, 850,900,950,1000,1050, 1100,1150,1200,1250, 1300,1350,1400,1450, 1500, 1550, 1600, 1650, 1700, 1750, 1800, 1850, 1900, 1950, 2000,2050, 2100, 2150, 2200, 2250, 2300, 2350, 2400,2450, 2500, 2550, 2600, 2650,2700, 2750, 2800, 2850, 2900, 2950, 3000, up to 3099 nucleotides for SEQ ID NO:1 or at least about 20, 25, 30, 35, 40, 45, 50, 55, 60, 65, 70, 75, 80, 85, 90, 95, 100, 150, 200, 250, 300, 350, 400, 450, 500, 550, 600, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, up to 1333 nucleotides for SEQ ID NO:3.

By “variants” is intended substantially similar sequences. For nucleotide sequences, conservative variants include those sequences that, because of the degeneracy of the genetic code, encode the amino acid sequence of one of the polypeptides of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode a nucleotide sequence of interest. Generally, variants of a particular nucleotide sequence of interest will have at least about 90%, 91%, 92%, 93%, 94%, 95%, 96%, 97%, and more preferably at least about 98%, 99% or more sequence identity to that particular nucleotide sequence as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, that is, a proliferating activity (for Evi1) or a differentiating activity (for PU.1) as described herein. Such variants may result from, for example, genetic polymorphism or from human manipulation. Biologically active variants of a native protein of the invention will have at least about 90%, 91%, 92%, 93%, 94%, 95%, 96%, 97%, and more preferably at least about 98%, 99% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants of the polypeptides of interest can be prepared by mutations in the DNA. Methods for mutagenesis and nucleotide sequence alterations are well known in the art. See, for example, Kunkel (1985) Proc. Nati. Acad. Sci. USA 82:488492; Kunkel et al. (1987) Methods in Enzymol. 154:367-382; U.S. Patent No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoffet al. (1978) Atlas of protein Sequence and Structure (Natl. Biomed. Res. Found., Washington, D.C.), herein incorporated by reference. Conservative substitutions, such as exchanging one amino acid with another having similar properties, may be preferable.

Thus, the genes and nucleotide sequences of the invention include both the naturally occurring sequences as well as mutant forms. Likewise, the proteins of the invention encompass both naturally occurring proteins as well as variations and modified forms thereof. Such variants will continue to possess the desired activity. Obviously, the mutations that will be made in the DNA encoding the variant must not place the sequence out of reading frame and preferably will not create complementary regions that could produce secondary MRNA structure. See, EP Patent Application Publication No. 75,444.

When it is difficult to predict the exact effect of the substitution, deletion, or insertion in advance of doing so, one skilled in the art will appreciate that the effect will be evaluated by routine screening assays. That is, the activity can be evaluated by suitable assays such as, but not limited to, proliferating activity assays or differentiating activity assays (described elsewhere herein).

Variant nucleotide sequences also encompass sequences derived from a mutagenic and recombinogenic procedure such as DNA shuffling. With such a procedure, one or more different promoter sequences or nucleotide sequences of interest can be manipulated to create a new sequence possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the nucleotide sequence of interest and other known myeloid modulating agents to obtain a new nucleotide sequence with an altered property of interest e.g. altered proliferating activity or altered differentiating activity. Strategies for such DNA shuffling are known in the art. See, for example, Stemmer (1994) Proc. Natl. Acad. Sci. 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. 94:4504-4509; Crameri et al. (1998) Nature 391:288-291; Miyazaki (2002) Nucleic Acids Research 30:E139-9; Song et al. (2002) Appl. Environ. Microbiol. 68:6146-51; Hayes et al. (2002) Proc. Natl Acad. Sci. 99:15926-31; Coco et al. (2001) Nature Biotechnol. 19:354-9; Kikuchi et al. (2000) Gene 243:133-7; and U.S. Pat. Nos. 5,606,793 and 5,837,458.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

(b) As used herein “comparison window” makes reference to a contiguous and specified segment of a polynucleotide sequence, wherein the polynucleotide sequence in the comparison window may comprise additions or deletions (i.e. gaps) compared to the reference sequence (which does not comprise additions or deletions) for optimal alignment of the two sequences. Generally the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Preferred, non-limiting examples of such mathematical algorithms are the algorithm of Myers and Miller (1988) CABIOS 4:11-17; the local homology algorithm of Smith et al. (1981) Adv. AppL. Math. 2:482; the homology alignment algorithm of Needleman and Wunsch (1970) J Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. For purposes of the present invention, comparison of nucleotide or protein sequences for determination of percent sequence identity to the sequences disclosed herein is preferably made using the GCG program GAP (Version 10.00 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program. Alignment may also be performed manually by inspection.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the finctional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman and Wunsch (1970) J Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

By “stably transformed” is intended that a myeloid cell has incorporated at least one copy of the transgene and is capable of transmitting a copy of the transgene to daughter cells, that the nuclear genome of a myeloid cell has incorporated at least one copy of the transgene, or that the nuclear genome of at least one cell of an animal of the invention has incorporated at least one copy of the transgene. By “transgene” is intended a nucleic acid molecule having a nucleotide sequence comprising either an expression cassette or a disruption cassette.

Transformation of pluripotent myeloid cells can be accomplished by any means known in the art including, but not limited to, retroviral transformation with the murine stem cell virus (DeKoter et al. (1998) EMBO J 17:4456-4468, herein incorporated by reference). Methods of transforming cells are widely known in the art and include, but are not limited to, microparticle bombardment, lipofection, transfection, retroviral transformation, include electroporation; transfection employing calcium chloride, rubidium chloride calcium phosphate, DEAE-dextran, or other substances; infection (where the vector is an infectious agent); and other methods. See generally, Sambrook et al. (2001) Molecular Cloning: A Laboratory Manual (3d ed., Cold Spring Harbor Laboratory Press, Plainview, N.Y.); Ausubel et al, eds. (2002) Current Protocols in Molecular Biology, Wiley-Interscience, New York, N.Y.; Katya & Freshney (1998) DNA Transfer to Cultured Cells John Wiley & Sons, New York, N.Y.; Freshney et al. (eds). (1994) Culture of Hematopoietic Cells John Wiley & Sons New York, N.Y. Introduction of nucleic acid molecules into cells is further discussed elsewhere herein. Reference to cells into which a transgene has been introduced is intended to also include the progeny of such cells.

Cells of the invention are transformed with an expression cassette comprising a promoter operably linked to a nucleotide sequence of interest. A number of promoters can be used in the practice of the invention. The promoters can be selected based on the desired outcome.

By “promoter” or “transcriptional initiation region” is intended a regulatory region of DNA usually comprising a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate. Thus, the promoter regions of use herein are generally further defined by comprising upstream regulatory elements such as those responsible for tissue and temporal expression of the coding sequence, enhancers and the like. Such elements are typically linked via a 5′ untranslated region, which may further modulate gene expression, to a coding region of interest. In the same manner, the promoter elements that enable expression in the desired tissue such as hematopoietic-tissue can be identified, isolated, and used with other core promoters to confirm hematopoietic-preferred expression. For genes in which the 5′ untranslated region does not affect cell specificity, alternative sources of 5′ untranslated leaders may be used in conjunction with these promoter elements.

By “heterologous nucleotide sequence” is intended a sequence that is not naturally occurring with the promoter sequence. While this nucleotide sequence is heterologous to the promoter sequence, it may be homologous, or native, or heterologous, or foreign, to the animal host.

It is recognized that to increase transcription levels or to alter tissue specificity, enhancers and/or tissue-preference elements may be utilized in combination with the promoters. For example, quantitative or tissue specificity upstream elements from other hematopoietic tissue-preferred nucleotide sequences may be combined with the promoter regions of the invention to augment hematopoietic tissue-preferred transcription. Such elements have been characterized, for example, p47(phox) and murine TRAP (Marden et al. (2003) Biochem Biophys Acta 1630:117-122; Walsh et al. (2003) Gene 307:111-123; herein incorporated by reference in their entirety).

Other enhancers are known in the art that would alter the tissue specificity by driving expression in other tissues in addition to hematopoietic tissue, such as in cardiac tissue, skeletal tissue, CNS tissue, pulmonary tissue, salivary tissue, lacrimal tissue, and vascular tissue, among others. These include, for example, upstream elements from the promoter of the aquaporin-5 promoter (Borok, et al. (2000) J Biol. Chem. 275:26507-14, herein incorporated by reference) that would give pulmonary and salivary-preferred expression in addition to hematopoietic tissue- preferred expression. Another example includes upstream elements from the human alpha-skeletal actin promoter, which would give expression in skeletal muscle, in addition to cardiac-preferred expression. The murine smooth muscle 22 α (SM22α) promoter is a smooth muscle cell preferred promoter (U.S. Pat. Nos. 6,015,711 and 5,837,534, herein incorporated by reference in their entirety). Further examples include sequences that allow cardiac preferred expression such as the murine TIMP-4 promoter A and B-type natriuretic peptide promoters, human cardiac troponin I promoter, mouse S100A1 promoter, salmon cardiac peptide promoter, GATA response element, inducible cardiac preferred promoters, rabbit β-myosin promoter, and mouse α-myosin heavy chain promoter (Rahkonen, et al. (2002) Biochim Biophys Acta 1577:45-52; Thuerauf and Glembotski (1997) J. Biol. Chem. 272:7464-7472; LaPointe et al (1996) Hypertension 27:715-722; Grepin et al. (1994) Mol. Cell Biol. 14:3115-29; Dellow, et al. (2001) Cardiovasc. Res. 50:3-6; Kiewitz, et al. (2000) Biochim Biophys Acta 1498:207-19; Majalahti-Palviainen, et al (2000) Endocrinology 141:731-740; Charron et al. (1999) Molecular & Cellular Biology 19:4355-4365; herein incorporated by reference in their entirety).

The regulatory sequences to which the polynucleotides described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, a Rous Sarcoma Virus promoter, an SV40 promoter, retrovirus long-terminal repeats, and the CMV constitutive promoter.

In other embodiments, the coding region is operably linked to an inducible regulatory element or elements. A variety of inducible promoter systems have been described in the literature and can be used in the present invention. These include, but are not limited to, tetracycline-regulatable systems (WO94/29442, WO 96/40892, WO96/01313, U.S. application Ser. No. 10/613,728); hormone responsive systems, interferon-inducible systems, metal-inducible systems, and heat-inducible systems, (WO93/20218); and ecdysone inducible systems. Some of these systems, including ecdysone inducible and tetracycline inducible systems are commercially available from Invitrogen (Carlsbad, Calif.) and Clontech (Palo Alto, Calif), respectively.

One of the most widely used conditional systems is the binary, tetracycline-based system, which has been widely used in both cells and animals to reversibly induce expression by the addition or removal of tetracycline or its analogues. (See Bujard (1999). J. Gene Med. 1:372-374; Furth, et al. (1994). Proc. Natl. Acad. Sci. USA 91:9302-9306; and Mansuy & Bujard (2000). Curr. Opin. Neurobiol. 10:593-596, herein incorporated by reference in their entirety.) Another example of such a binary system is the cre/loxP recombinase system of bacteriophage P1. For a description of the cre/loxP recombinase system, see, e.g., Lakso et al. (1992) PNAS 89:6232-6236. In the Cre/LoxP recombinase system, the activator transgene encodes recombinase. If a cre/loxP recombinase system is used to regulate expression of the transgene, animals containing transgenes encoding both the Cre recombinase and a selected target protein are required. Another example of a recombinase system is the FLP recombinase system of S. cerevisiae (O'Gorman et al. (1991) Science 251:1351-1355.

By “inducible” is intended that a chemical stimulus alters expression of the operably linked nucleotide sequence of interest by at least 1%, 5%, preferably 10%, 20%, more preferably 30%, 40%, 50%, 60%, 70%, 80%, 90%, 99% or more. The difference may be an increase or decrease in expression levels. Methods for assaying expression levels are described elsewhere herein. The chemical stimulus may be administered or withdrawn. Various chemical stimuli are known in the art. In an embodiment, the chemical stimulus is tetracycline, or an analog thereof.

It is recognized that a range of expression of the heterologous nucleotide sequence may be use in the invention. Thus, the nucleotide sequences of interest may be operably linked to weak promoters or strong promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts; conversely, a strong promoter drives expression of a coding sequence at a high level, or at about 1/10 transcripts to about 1/100 transcripts to about 1/1000 transcripts.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

Such expression cassettes will comprise a transcriptional initiation region comprising a promoter nucleotide sequence operably linked to the heterologous nucleotide sequence whose expression is to be controlled by the promoter. Such an expression cassette is provided with at least one restriction site for insertion of the nucleotide sequence of interest to be under the transcriptional regulation of the regulatory regions. The expression cassette may additionally contain selectable marker genes.

The expression cassette will include in the 5′-to-3′ direction of transcription, a transcriptional and translational initiation region, and a heterologous nucleotide sequence of interest. In addition to containing sites for transcription initiation and control, expression cassettes can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al. (1989) Molecular Cloning: A Laboratory Manual 2nd. ed., Cold Spring Harbor Laboratory Press, Cold Spring Harbor, N.Y.).

The expression cassette comprising a promoter operably linked to a heterologous nucleotide sequence of interest may also contain at least one additional nucleotide sequence for a gene to be co-transformed into the organism. Alternatively, the additional sequence(s) can be provided on another expression cassette.

Where appropriate, the heterologous nucleotide sequence whose expression is to be under the control of a promoter sequence and any additional nucleotide sequence(s) may be optimized for increased expression in the transformed cell or transformed animal. That is, these nucleotide sequences can be synthesized using species preferred codons for improved expression, such as rabbit-preferred codons for improved expression in rabbits or mouse-preferred codons in mice. Methods are available in the art for synthesizing species-preferred nucleotide sequences. See, for example, Wada et al. (1992) Nucleic Acids Res. 20 (Suppl.), 2111-2118; Butkus et al. (1998) Clin Exp Pharnacol Physiol SuppL. 25:S28-33; and Sambrook et al. (1989) Molecular Cloning: A Laboratory Manual 2nd. ed., Cold Spring Harbor Laboratory Press, Cold Spring Harbor, N.Y., herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the heterologous nucleotide sequence may be adjusted to levels average for a given cellular host, as calculated by reference to known genes expressed in the host cell. In an embodiment, the sequence is modified to avoid predicted hairpin secondary mRNA structures. In an embodiment the sequence is modified to yield hairpin RNA structures for use in siRNA.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Nat. Acad. Sci. USA 86:6126-6130); potyvirus leaders, for example, TEV leader (Tobacco Etch Virus) (Allison et al. (1986)); MDMV leader (Maize Dwarf Mosaic Virus) (Virology 154:9-20); and human immunoglobulin heavy-chain binding protein (BiP) (Macejak et al. (1991) Nature 353:90-94). Other methods known to enhance translation and/or mRNA stability can also be utilized, for example, introns, and the like.

In those instances where it is desirable to have the expressed product of the heterologous nucleotide sequence directed to a particular organelle, particularly the mitochondria, the nucleus, the endoplasmic reticulum, the Golgi apparatus; or secreted at the cell's surface or extracellularly; the expression cassette may further comprise a coding sequence for a transit peptide. Such transit peptides are well known in the art and include, but are not limited to, the transit peptide for the acyl carrier protein, the small subunit of RUBISCO, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose; in vitro mutagenesis; primer repair; restriction; annealing; substitutions, for example, transitions and transversions; or any combination thereof may be involved.

Selectable marker genes for selection of transformed cells or tissues can include genes that confer antibiotic resistance. Examples of suitable selectable marker genes include, but are not limited to, genes encoding resistance to neomycin (Schwartz et al (1991) Proc. Natl. Acad. Sci. 88: 10416-20); chloramphenicol (Herrera Estrella et al. (1983) EMBO J. 2:987-992); methotrexate (Herrera Estrella et al. (1983) Nature 303:209-213; Meijer et al. (1991) Plant Mol. Biol. 16:807-820); hygromycin (Waldron etal. (1985) Plant Mol. Biol. 5:103-108; Zhijian etal. (1995) Plant Science 108:219-227); streptomycin (Jones et al. (1987) Mol. Gen. Genet. 210:86-91); spectinomycin (Bretagne-Sagnard et al. (1996) Transgenic Res. 5:131-137); bleomycin (Hille et al. (1990) Plant MoL Biol. 7:171-176); sulfonamide (Guerineau et al. (1990) Plant Mol. Biol. 15:127-136); puromycin (Abbate et al (2001) Biotechniques 31:336-40; cytosine arabinoside (Eliopoulos et al. (2002) Gene Ther. 9:452-462); 6-thioguanine (Tucker et al. (1997) Nucleic Acid Research 25:3745-46).

Other genes that could serve utility in the recovery of transgenic events but might not be required in the final product would include, but are not limited to, examples such as GUS (b-glucoronidase; Jefferson (1987) Plant Mol. Biol. Rep. 5:387); GFP (green fluorescence protein; Wang et al. (2001) Anim Biotechnol 12:101-110; Chalfie et al. (1994) Science 263:802), BFP (blue fluorescence protein; Yang et al. (1998) J BioL Chem. 273:8212-6), CAT; and luciferase (Riggs et al. (1987) Nucleic Acid Res. 15 (19):8115; Luchrsen et al. (1992) Methods Enzymol. 216: 397-414).

Delivery vehicles suitable for incorporation of a polynucleotide for introduction into a host cell include, but are not limited to, viral vectors and non-viral vectors (Verma and Somia (1997) Nature 389:239-242).

A wide variety of non-viral vehicles for delivery of a polynucleotide are known in the art and are encompassed in the present invention. An isolated nucleic acid molecule can be delivered to a cell as naked DNA (WO97/40163). Alternatively, a polynucleotide can be delivered to a cell associated in a variety of ways with a variety of substances (forms of delivery) including, but not limited to, cationic lipids; biocompatible polymers, including natural and synthetic polymers; lipoproteins; polypeptides; polysaccharides; lipopolysaccharides; artificial viral envelopes; metal particles; and bacteria. A delivery vehicle can be a microparticle. Mixtures or conjugates of these various substances can also be used as delivery vehicles. A polynucleotide can be associated non-covalently or covalently with these forms of delivery. Liposomes can be targeted to a particular cell type, e.g., to a myeloid cell.

Non-viral delivery vehicles comprising a polynucleotide can be introduced into host cells and/or target cells by any method known in the art, such as transfection by the calcium phosphate coprecipitation technique; electroporation; electropermeablization; liposome-mediated transfection; ballistic transfection; biolistic processes including microparticle bombardment, jet injection, and needle and syringe injection, or by microinjection. Numerous methods of transfection are known to the skilled worker in the field.

Viral vectors include, but are not limited to, DNA viral vectors such as those based on adenoviruses, herpes simplex virus, poxvirus such as vaccinia virus, and parvoviruses, including adeno-associated virus; and RNA viral vectors, including but not limited to, the retroviral vectors. Retroviral vectors include murine leukemia virus, murine stem cell virus, and lentiviruses such as human immunodeficiency virus. See Naldini et al. (1996) Science 272:263-267.

Viral delivery vectors can be introduced into cells by infection. Alternatively, viral vectors can be incorporated into any of the non-viral delivery vectors described above for delivery into cells. For example, viral vectors can be mixed with cationic lipids (Hodgson and Solaiman (1996) Nature Biotechnol. 14:339-342); or lamellar liposomes (Wilson et al. (1977) Proc. Natl. Acad. Sci. 74:3471-3475; and Faller et al. (1984) J Virol. 49:269-272).

For in vivo delivery, the vector can be introduced into an individual or organism by any method known to the skilled artisan.

A substance having “proliferating activity” stimulates proliferation directly or indirectly. By “proliferation” is intended a mitotic increase in cell number or an increase in the average size of the cell. For instance, the substance may stimulate proliferation directly or the substance may stimulate proliferation indirectly through activation of a pathway capable of stimulating cell proliferation, particularly myeloid cell proliferation. Progeny cells remain as pluripotent as the mother cell. Methods of assaying proliferation include, but are not limited to, DNA synthesis assays, tritiated thymidine uptake assays, morphological assessment, respiration assays, metabolic assays, colony forming unit assays, and cytological quantification.

A substance having “differentiating activity” stimulates differentiation directly or indirectly. By “differentiation” is intended the progression of a pluripotent cell to a cell type from which fewer cell types can emerge. Such differentiation can occur at any time during the cell cycle. By intermediately differentiated is intended that at least one characteristic of a terminally differentiated cell is present and that one or more characteristics of a terminally differentiated cell is not present or is below the level typical of a terminally differentiated cell. Characteristics of differentiated cells include, but are not limited to, expression of CD4, CD8, CD3ε, CD5, B220, Gr-1, Ter119, and other differentiation markers, anchorage independent proliferation, anchorage dependent proliferation, proliferation rate, catabolism of surfactant proteins, catabolism of surfactant lipids, expression of pathogen associated molecular pattern receptors (for example, toll-like receptors or the mannose receptor), pathogen phagocytosis, intracellular bacteria killing, pathogen stimulated cytokine receptor (tumor necrosis factor α, interleukin-12, and interleukin- 18), and Fc receptor mediated phagocytosis. Any method of assaying a differentiation characteristic may be used to monitor differentiation of the cells. Methods of inducing differentiation are known in the art and include, but are not limited to, conditioning medium from pokeweed mitogen stimulated spleen cells, and IL-3 treatment.

An embodiment of the invention provides a process of modulating Evi1 expression in a cell, particularly a myeloid cell. The process involves isolating one or more myeloid cells, and transforming the cell or cells with a first expression cassette comprising an Evi1 nucleotide sequence or a fragment or variant thereof. In an aspect of the process, a cell is also transformed with a second expression vector comprising a PU.1 nucleotide sequence or fragment or variant thereof. It is envisioned that transformation with the first and second expression cassettes occurs simultaneously or consecutively. Further it is recognized that a second expression cassette comprising PU.1 may transform the cell prior to a first expression cassette comprising Evi1. Further it is understood that the Evi1 expression cassette and the PU.1 expression cassette may be present on the same nucleic acid molecule or may be present on separate nucleic acid molecules.

By “modulating Evi1 expression levels” is intended that the expression of the Evi1 nucleotide sequence in a transgenic cell of the invention differs from expression levels in a non-transgenic cell by at least 1%, 2%, 3%, 4%, 5%, 10%, 15%, 20%, 25%, 30%, 35%, 40%, 45%, 50%, 55%, 60%, 65%, 70%, 75%, 80%, 90%, 95%, or 100%. The difference may be an increase or decrease in expression levels. It is recognized that altered Evi1 expression levels may be constitutive or variable depending in part on the type of promoter and exogenous PU.1 levels.

Methods of assaying expression levels are known in the art and include, but are not limited to, qualitative Western blot analysis, immunoprecipitation, radiological assays, polypeptide purification, spectrophotometric analysis, Coomassie staining of acrylamide gels, ELISAs, RT-PCR, 2-D gel electrophoresis, microarray analysis, in situ hybridization, chemiluminescence, silver staining, enzymatic assays, ponceau S staining, multiplex RT-PCR, immunohistochemical assays, radioimmunoassay, colorimetric analysis, immunoradiometric assays, positron emission tomography, Northern blotting, fluorometric assays, fluorescence activated cell sorter staining of permeabilized cells, radioimmunosorbent assays, real-time PCR, hybridization assays, sandwich immunoassays, flow cytometry, SAGE, differential amplification, or electronic analysis. See, for example, Ausubel et al, eds. (2002) Current Protocols in Molecular Biology, Wiley-Interscience, New York, N.Y.; Coligan et al (2002) Current Protocols in Protein Science, Wiley-Interscience, New York, N.Y.; Sun et al. (2001) Gene Ther. 8:1572-1579; de Jager et al. (2003). Clin. & Diag. Lab. Immun. 10:133-139; U.S. Pat. Nos. 6,489,4555; 6,551,784; 6,607,879; 4,981,783; and 5,569,584; herein incorporated by reference in their entirety.

An embodiment of the invention provides a process of modulating myeloid cell differentiation. The process involves isolating one or more myeloid cells, transforming the cell or cells with a first expression cassette comprising an Evi1 nucleotide sequence or a fragment or variant thereof, and monitoring differentiation of the cell or cells. In an aspect of the process, a cell is also transformed with a second expression vector comprising a PU.1 nucleotide sequence or fragment or variant thereof. It is envisioned that progeny cells may also be monitored for differentiation. Methods of monitoring differentiation are known in the art. Further it is recognized that a second expression cassette comprising PU.1 may transform the cell prior to a first expression cassette comprising Evi1. Further it is understood that the Evi1 expression cassette and the PU.1 expression cassette may be present on the same nucleic acid molecule or may be present on separate nucleic acid molecules.

Propagation of mammalian cells in culture is well known. See, Tissue Culture, Academic Press, Kruse and Patterson, ed. (1973); Freshney, Ian Ed. (ed) (2000) Culture of Animal Cells: A Manual of Basic Technique (4^{th} ed) John Wiley & Sons. New York, N.Y.; Masters, John R. W. (ed) Animal Cell Culture (3^{rd} ed) Oxford University Press: Great Britain, herein by reference. Cell lines are widely used as in vitro models for studying the events involved in in vivo cellular development or disorders. Typically cells obtained from primary culture of healthy tissue undergo a finite number of divisions and then cease to divide. The number of divisions that primary culture cells undergo depends on the cell type, the age of the subject from which the cells were obtained, and various other factors. Two major properties of a cell line are the cells' capability to proliferate and to differentiate. These properties are of particular interest in disorders that involve pluripotent stem cells. Prior to this invention long-term maintenance of undifferentiated myeloid cells such as macrophages in culture has not been feasible.

An embodiment of the invention provides methods of preparing a myeloid cell line suitable for tissue culture. The method involves isolating myeloid cells, transforming the cells with an expression cassette comprising an Evi1 nucleotide sequence or a fragment or variant thereof, and selecting cells comprising the expression cassette. Methods of selecting cells comprising an expression cassette are known in the art and include, but are not limited to, the use of selectable markers such as antibiotic resistance genes and metabolic genes. It is understood that selection may occur continuously, discontinuously, or in one event. In an aspect of the invention, the method further comprises obtaining a clonal population. A clonal population of cells, as used herein, is a population of two or more cells which have one or more of the following properties: they share a common stem cell ancestor; they share a common pluripotent myeloid cell ancestor; they share a common intermediately differentiated myeloid cell ancestor; or they share a common transfected ancestor. Methods of obtaining a clonal population are known in the art and include, but are not limited to, serial dilution and single cell expansion. In an aspect of the process, a cell is also transformed with a second expression vector comprising a PU.1 nucleotide sequence or fragment or variant thereof.

An embodiment of the invention provides processes of preparing an immortalized myeloid cell line. By “immortalized” is intended that a cell of the cell line will, if maintained in a suitable environment, divide at least 10, 15, or 20 times, preferably 30, 40, 50, or 60 times, more preferably 70, 80, 90, or 100 times, still more preferably 110, 120, 130, 140, or 150 times, yet still more preferably at least 160, 170, 180, 190 or 200 times. The process involves isolating myeloid cells, transforming the cells with an expression cassette comprising an Evi1 nucleotide sequence or a fragment or variant thereof, selecting cells comprising the expression cassette, and maintaining the cells. Methods of maintaining cells in tissue culture are known in the art. Cells may be maintained by providing suitable culture media, adequate gas exchange, and routinely passaging the cells. Additionally the cells may be maintained by preparing the cells for long term storage, and storing the cells. Methods of storing cells are known in the art and include for example, suspending the cells in glycerol or a similar substance, flash freezing, and storing the cells in liquid nitrogen or −80° C. (Ausubel et al., eds. (1995) Current Protocols in Molecular Biology, Chapter 2 (Greene Publishing and Wiley-Interscience, New York).

An embodiment of the invention provides processes of modulating differentiation of a myeloid cell line. The process involves isolating myeloid cells, transforming the cells with an expression cassette comprising an Evi1 nucleotide sequence or a fragment or variant thereof, selecting cells comprising the expression cassette, and monitoring differentiation of the cells. A further embodiment of the invention provides processes of modulating differentiation of a myeloid cell line. The process involves isolating myeloid cells, transforming the cells with a first expression cassette comprising an Evi1 nucleotide sequence or a fragment or variant thereof, transforming the cells with a second expression cassette comprising a PU.1 nucleotide sequence or a fragment or variant thereof, and monitoring differentiation of the cells.

The invention provides methods of assaying the differentiating activity, the immune disorder modulating capabilities, or immune disorder treating activity of a compound of interest. The method involves providing a myeloid cell or cells comprising an expression cassette comprising an Evi1 nucleotide sequence or a fragment or variant thereof, administering a compound of interest to the cell, and monitoring the differentiation of the cell or an immune disorder associated phenotype. The invention particularly provides methods of assaying the ability of a compound to affect differentiation of a myeloid cell into a cell type such as a macrophage, neutrophil, or mast cell. Methods of monitoring differentiation or immune disorder associating phenotypes are described elsewhere herein.

To assay the differentiating activity, the immune disorder modulating capability, or immune disorder treating activity of a compound, multiple transgenic cells of the invention, e.g. at least a first and second transgenic cell, are provided. The terms “first,” “experimental,” or “test” transgenic cell refer to a transgenic cell to which a compound of interest is administered. The terms “second” or “control” transgenic cell refer to a transgenic cell to which a placebo is administered. In an embodiment, the first and second transgenic cells are clonal and subject to similar environmental conditions. In an embodiment, more than one cell may be a first transgenic cell. In an embodiment more than one cell may be a second transgenic cell.

After administration of either the compound of interest or the placebo, the first and second transgenic cells are incubated for a period of time. The period of time will have a predetermined duration appropriate to analysis of the differentiating activity or disorder associated phenotype. Such durations include, but are not limited to, 30 seconds; 1, 5, 10, 30, or 60 minutes; 8, 12, 24, 36, or 48 hours; 3, 4, 5, 6, or 7 days; 2,3, or 4 weeks; 2,3,4,5,6,7,8,9, 10, 11, or 12 months; up to 3 years. Monitoring of a differentiating activity or disorder associated phenotype may occur continuously; at a single interval; or at multiple intervals, such as, but not limited to, hourly, daily, weekly, and monthly. Any method of assaying a differentiating activity or disorder associated phenotype known in the art may be used to monitor the effects of the compound of interest on a transgenic cell of the invention.

The invention provides methods of assaying the capability of a compound of interest to treat an immune disorder. By “treat” is intended to cure, heal, alleviate, relieve, alter, remedy, ameliorate, improve, or affect the disease or symptoms of the disease in a patient who has an immune disorder or symptom of an immune disorder.

By immune disorder is intended any disease or disorder relating to the immune system including, but not limited to, polycythemia vera, myelofibrosis, myeloid metaplasia, primary thrombocythemia, chronic myelogenous leukemia, acute myelogenous leukemia, relative erythrocytosis, spurious erythrocytosis, Gaisbock's syndrome, stress polycythemia, agnogneic myeloid metaplasia, secondary thrombocythemia, rheumatoid arthritis, inflammatory bowel disease, tuberculosis, sarcoidosis, Wegener's granulomatosis, leukopenia, neutropenia, granulocytopenia, agranulocytosis, cyclic neutropenia, severe congenital neutropenia, Kostmann syndrome, idiopathic neutropenia, myelodysplasia, Schwachman-Diamond syndrome, cartilage hair hypoplasia, dyskeratosis congenital, glycogen storage disease type IB, eosinophilia, idiopathic hypereosinophilic syndrome, disseminated eosinophilic collagen disease, eosinophilic leukemia, Loffler's fibroplastic endocarditis with eosinophilia, histiocytic syndromes, Langerhans cell histiocytosis, histiocytosis X, eosinophilic granuloma, Letterer-Siwe disease, Hand-Schuller-Christian disease, myelodysplastic syndromes, acute myelogenous leukemia, acute myeloid leukemia, acute myelocytic leukemia, acute promyelocytic leukemia, chronic myeloid leukemia, chronic myelocytic leukemia, chronic granulocytic leukemia, preleukemia, refractory anemias, Ph-negative chronic myelocytic leukemia, chronic myelomonocytic leukemia, and agnogenic myeloid metaplasia.

Immune disorders of particular interest include but are not limited to, myeloid disorders, erythroid disorders, leukemias, polycythemia vera, myelofibrosis, myeloid metaplasia, primary thrombocythemia, chronic myelogenous leukemia, acute myelogenous leukemia, relative erythrocytosis, spurious erythrocytosis, Gaisbock's syndrome, stress polycythemia, agnogneic myeloid metaplasia, leukopenia, neutropenia, granulocytopenia, agranulocytosis, cyclic neutropenia, severe congenital neutropenia, Kostmann syndrome, idiopathic neutropenia, myelodysplasia, myelodysplastic syndromes, acute myelogenous leukemia, acute myeloid leukemia, acute myelocytic leukemia, acute promyelocytic leukemia, chronic myeloid leukemia, chronic myelocytic leukemia, chronic granulocytic leukemia, preleukemia, refractory anemias, Ph-negative chronic myelocytic leukemia, chronic myelomonocytic leukemia, and agnogenic myeloid metaplasia.

Immune disorder associated phenotypes include adherence, cell surface antigens, morphology, cell adhesion, complement mediated phagocytosis, antibody mediated phagocytosis, pathogen destruction, cell growth, secretion, differentiation, phagocytosis, survival, cellular morphology, leukocyte alkaline phosphatase assays, platelet aggregation, giant platelets, megakaryocyte fragmentation, hypogranulation, vacuolation, terminal transferase stains, myeloperoxidase stains, Sudan black B, and specific and nonspecific esterase histochemical stains, bcr rearrangement, and presence of Philadelphia chromosome, myeloid cytokine receptor subunit expression (myeloid cytokine receptor subunits include, but are not limited to, G-CSFR, GM-CSFRα, GM-CSFRβ_{c}, c-Fms), myeloid structural gene expression (myeloid structural genes include, but are not limited to, myeloperoxidase, CD16, CD32, CD64, CD11b, macrophage scavenger receptor), chloro-acetate esterase expression, AA4.1 expression, proliferation, rosette formation, CD64, CD68, MAX 1, HLADR, CD71, expression of differentiation markers, and proliferation rate.

Immune disorder associated phenotype assays include, but are not limited to, methylcellulose colony forming assays, RT-PCR, phase contrast microscopy, Wright staining, flow cytometry, morphological analysis, Western blot, DNA synthesis assays, tritiated thymidine incorporation assays, and fluorescent latex bead phagocytosis assays (Berclaz et al. (2002) Blood 100:4193-4200, herein incorporated by reference).

The invention provides methods of identifying anti-leukemia agents. The method involves providing a myeloid cell or cells comprising an expression cassette comprising an Evi1 nucleotide sequence or a fragment or variant thereof, administering a compound of interest to the cell, and monitoring a leukemia associated phenotype of said cell.

To identify anti-leukemia agents, multiple transgenic cells of the invention, e.g. at least a first and second transgenic cell, are provided. The terms “first,” “experimental,” or “test” transgenic cell refer to a transgenic cell to which a compound of interest is administered. The terms “second” or “control” transgenic cell refer to a transgenic cell to which a placebo is administered. In an embodiment, the first and second transgenic cells are clonal and subject to similar environmental conditions. In an embodiment, more than one cell may be a first transgenic cell. In an embodiment more than one cell may be a second transgenic cell. It is understood that a transgenic cell useful in the method of the invention may be contained within a transgenic animal of the invention.

After administration of either the compound of interest or the placebo, the first and second transgenic cells are incubated for a period of time. The period of time will have a predetermined duration appropriate to analysis of the leukemia associated phenotype. Such durations include, but are not limited to, 30 seconds; 1, 5, 10, 30, or 60 minutes; 8, 12, 24, 36, or 48 hours; 3, 4, 5, 6, or 7 days; 2, 3, or 4 weeks; 2, 3, 4, 5, 6, 7, 8, 9, 10, 11, or 12 months; up to 3 years. Monitoring of a leukemia associated phenotype may occur continuously; at a single interval; or at multiple intervals, such as, but not limited to, hourly, daily, weekly, and monthly. Any method of assaying a leukemia associated phenotype known in the art may be used to monitor the effects of the compound of interest on a transgenic cell of the invention.

Leukemia phenotypes include, but are not limited to, weakness, headache, lightheadedness, visual disturbances, dyspnea, bleeding diasthesis, hepatomegaly, splenomegaly, bone pain, ulcers, thrombosis, anisocytosis, poikilocytosis, arterial blood gas measurements, vitamin B12 levels, vitamin B12 binding, digital ischemia, epistaxis, GI bleeding, platelet aggregation, giant platelets, megakaryocyte fragmentation, Auer rod development, Philadelphia chromosome presence, bcr gene rearrangement, adherence, cell surface antigens, morphology, cell adhesion, complement mediated phagocytosis, antibody mediated phagocytosis, pathogen destruction, cell growth, secretion, differentiation, phagocytosis, survival, cellular morphology, leukocyte alkaline phosphatase assays, hypogranulation, vacuolation, terminal transferase stains, myeloperoxidase stains, Sudan black B, and specific and nonspecific esterase histochemical stains, myeloid cytokine receptor subunit expression (myeloid cytokine receptor subunits include, but are not limited to, G-CSFR, GM-CSFRα, GM-CSFRβ_{c}, c-Fms), myeloid structural gene expression (myeloid structural genes include, but are not limited to, myeloperoxidase, CD16, CD32, CD64, CD11b, macrophage scavenger receptor), chloro-acetate esterase expression, AA4.1 expression, proliferation, rosette formation, CD64, CD68, MAX 1, HLADR, CD71, expression of differentiation markers, and proliferation rate.

Leukemia phenotype assays include, but are not limited to, platelet count, white blood cell count, white blood cell differential, arterial blood gas measurement, urinalysis, sonography, CT, leukocyte alkaline phosphatase assays, chromium labeling, vitamin B12 binding assays, peripheral blood smears, chest X-rays, ECG, liver function tests, kidney function tests, histochemical studies, cytogenetics, immunophenotyping, Southern blotting, expression analysis, methylcellulose colony forming assays, RT-PCR, phase contrast microscopy, Wright staining, flow cytometry, morphological analysis, Western blot, DNA synthesis assays, tritiated thymidine incorporation assays, and fluorescent latex bead phagocytosis assays (Berclaz et al. (2002) Blood 100:4193-4200, herein incorporated by reference).

The term “administer” is used in its broadest sense and includes any method of introducing a compound into a myeloid cell of the present invention. This includes producing polypeptides or polynucleotides in vivo as by transcription or translation in vivo of polynucleotides that have been exogenously introduced into a subject. Thus, polypeptides or nucleic acids produced in the subject from the exogenous compositions are encompassed in the term “administer.”

A “compound” comprises, but is not limited to, nucleic acid molecules, small interfering RNAs, peptides, polypeptides, small molecules, glycoproteins, antisense nucleotide sequences, peptidomimetics, lipids, antibodies, receptor inhibitors, ligands, sterols, steroids, hormones, kinases, kinase inhibitors, agonists, antagonists, diuretics, enzymes, enzyme inhibitors, carbohydrates, deaminases, deaminase inhibitors, G-proteins, G-protein receptor inhibitors, calcium channel modulators, hormone receptor modulators, alcohols, phosphatases, lactones, neurotransmitter inhibitors, neurotransmitter receptor modulators, negative inotropic agents, β-blockers, Ca²⁺ antagonists, transcription factors, Evi1, and PU.1. A compound may additionally comprise a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Anti-leukemia agents identified by the methods of this invention may be used in the treatment of human individuals. Embodiments of the invention provide methods of treating an immune disorder, particularly a myeloid disorder, more particularly a leukemia. The methods comprise administering a therapeutically effective amount of a anti-leukemia agent to a subject exhibiting a disorder of interest. Anti-leukemia agents useful in the treatment of a disorder discussed herein are provided in therapeutically effective amounts. By “therapeutically effective amounts” is intended an amount sufficient to modulate the desired response. Appropriate therapeutically effective amounts vary depending upon the actual anti-leukemia agent utilized, the delivery mode, and the agent's mode of action.

In an embodiment, the invention provides methods of modulating the transition from the proliferation of undifferentiated macrophage lineage cells to differentiated macrophages. The method comprises the steps of isolating macrophage lineage cells, transforming the cells with an expression cassette comprising an Evi1 nucleotide sequence, and monitoring the differentiation of the cells. By “macrophage lineage cell” is intended a cell capable of differentiating into a macrophage. In an aspect of the method, the invention further comprises the step of transforming the cells with a second expression cassette comprising PU.1 or a fragrnent or variant thereof. In an aspect of the invention, the differentiated macrophages are terminally differentiated. In an aspect of the invention, the differentiated macrophages are intermediately differentiated. It is recognized that Evi1 and PU.1 can be used to modulate the differentiation state of a cell causing the cell to become less or more differentiated.

The following examples are offered by way of illustration and not limitation.

### EXPERIMENTAL

### Example 1

### Analysis of PU.1 Effect on Evi-1 Expression

An immature macrophage cell line (mAM) and a terminally differentiated macrophage cell line (MH-S) were examined for Evi-1 expression. The mAM cell line is a murine alveolar macrophage cell line with an immature phenotype and restricted PU.1 expression. The mAM cell line was obtained from a GM-CSF−/−mouse and is intermediately differentiated (Shibata et al. (2001) Immunity 15:557-567 and Berclaz et al. (2002) Blood 100:4193-4200, herein incorporated by reference in their entirety). MH-S cells (American Type Culture Collection, CRL-2019, Manassas Va.) express PU.1 normally and are differentiated (Mbawuike and Herscowitz, (1989) J Leukoc. Biol. 46:119-127, herein incorporated by reference in its entirety).

The p50MX-Neo retroviral vector was digested with the NotI restriction enzyme. The full length Evi-1 cDNA (SEQ ID NO:1, Genbank Accession No. M21829) was digested with Not1. The digested retroviral vector and Evi-1 were incubated together in the presence of ligase to yield p50MX-Neo-Evi-1. In p50MX-Neo-Evi-1, Evi-1 is operably linked to the myeloproliferative virus long terminal repeat. The p50MX-Neo-Evi-1 retroviral vector was used to transduce mAM and MH-S cells. mAM cells were transduced with retroviral expression cassettes comprising PU.1 (Shibata et al. (2001) Immunity 15:557-567). A third mAM cell line was generated by transduction with both Evi1 and PU.1 retroviral expression cassettes. MS-H cells were transduced with retroviral expression cassettes comprising PU.1, Evi1, or both the Evi1 and PU.1 retroviral expression cassettes (Shibata et al. (2001) Immunity 15:557-567 and Berclaz et al. (2002) Blood 100:4193-4200, herein incorporated by reference in their entirety).

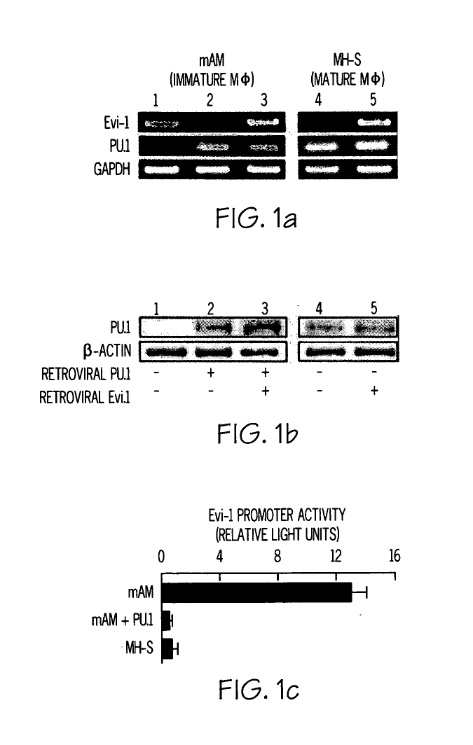

Total RNA was isolated from mAM, mAM PU.1, mAM PU.1+Evi1, MS-H, and MS-H+Evi1 cells. RT-PCR reactions were performed on the RNA. Primer sets specific to endogenous PU.1, retroviral PU.1, endogenous Evi-1, and GAPDH were used. All RT-PCR analyses were performed in duplicate in two separate experiments. Products of the RT-PCR were resolved on an agarose gel. Results from a typical experiment are presented in FIG. 1, panel A.

AM, mAM PU.1, mAM PU.1+Evi1, MS-H, and MS-H+Evi1 cells were lysed. Aliquots of the cell lysates were electrophoresed on SDS-polyacrylamide gels. After electrophoresis, the polypeptides were transferred to a nitrocellulose membrane. The membranes were blocked, then incubated with anti-PU.1 antibodies or β-actin antibodies. After washing, the membranes were incubated with secondary antibodies and the products were visualized. Western blot analyses were performed twice. Results of a typical experiment are shown in FIG. 1, panel B.

### Example 2

### Evi-1 Promoter Response to PU.1

Murine genomic DNA was incubated with oligonucleotide primers specific to regions of the 5′ portion of exon I and adjacent 5′ sequence. The amplified product was digested with EcoRI and SstI to yield a 5 kb fragment (GenBank Accession No. AL929377). The pGL2 Basic firefly luciferase reporter plasmid strain was digested with EcoRI and Sstl. The digested Evi1 promoter and pGL2 plasmid were incubated together with ligase to yield pGL2E5.

mAM, MS-H, and mAM+PU.1 cells were plated at 10⁵ per 35 mm plate and incubated for one day. Then the mAM, MS-H, and mAM+PU.1 cells were transformed with the pGL2E5 plasmid and a control plasmid comprising a renilla luciferase reporter. Plasmids were transfected into cells using Effectine (QIAGEN, Valencia Calif.). The transfected cells were incubated an additional 48 hours. Firefly and renilla luciferase activities were quantified on a Monolight 3010 luminometer (Analytical Luminescence Laboratory, Ann Arbor Mich.) using the dual luciferase assay kit (Promega, Madison Wis.). Firefly luciferase values were normalized for renilla luciferase activity. The data shown in FIG. 1, panel C represent the mean of at least 5 determinations for each cell line and are typical results.

### Example 3

### Macrophage Proliferation Assay

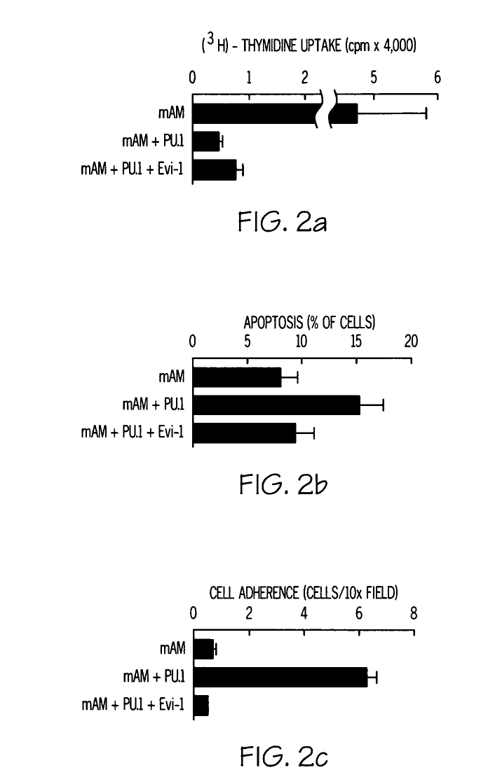

Proliferation of mAM, mAM+PU.1, and mAM+PU.1+Evi1 cells was assessed by determining the extent of DNA synthesis using a [³H]-thymidine incorporation assay. [³H]-thymidine incorporation assays are known in the art and described in Kurokawa, M et al. (1998) Nature 394:92-96, herein incorporated by reference in its entirety. The data shown in FIG. 2 represent the mean of 9 determinations for each cell line.

### Example 4

### Apoptosis Assessment

AM, mAM+PU.1, and mAM+PU.1+Evi1 cells were cultured in 24 well plates (5×10⁵/well) in DMEM in the absence or presence of 1% fetal calf serum. Culture media was replaced after 24 hours, and the cells were maintained for 48 hours. The cells were washed in phosphate buffered saline and collected in cell-dissociation buffer (10 mM HEPES/NaOH [pH 7.4], 140 mM NaCl, 2.5 mM CaCl₂). 7AAD (Pharmigen, San Diego, Calif.) was used rather than propidium iodide (Yang et al. (2000) Immunity 12:557-568). A FACScan flow cytometer (Becton Dickinson, San Jose Calif.) was used to perform 4 color FACS to quantify the percentage of cells undergoing apoptosis. Data shown in FIG. 2 represent the mean of at least 5 determinations for each line.

### Example 5

### Cell Adherence Assay

Quantification of cell adhesion of mAM, mAM+PU.1, and mAM +PU.1+Evi1 cells was performed using ultra low adherence plastic culture dishes (Costar, Corning N.Y.) as described in Shibata et al. (2001) Immunity 15:557-567. Data shown in FIG. 2 represent the mean of at least 7 determinations for each line.

### Example 6

### Non-specific and FcγR-mediated Phagocytosis Assays

Fluorescein-isothiocyanate (FITC)-labeled latex microspheres (2 μm dia. From Spherotech, Libertyville Ill.) were incubated with bovine serum albumin (fraction V, Sigma, St. Louis Mo.) at 10 mg/ml for 60 minutes at 37° C. The beads were washed 3 times and resuspended in phosphate buffered saline at a concentration of 1.5×10⁹ beads per milliliter. IgG-opsonized beads were prepared by incubating albumin coated beads with anti-albumin antibody (Pharmingen) at 1500 μ/mL for 30 minutes at 37° C. The beads were washed 3 times and resuspended in phosphate buffered saline at a concentration of 1.5×10⁹ beads per milliliter.

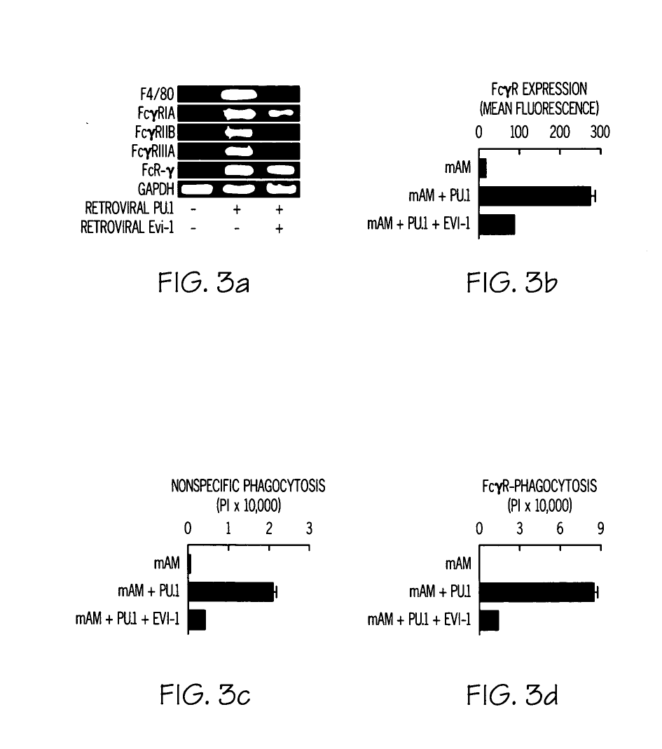

The macrophage cells were plated in 35-mm plates at 10⁵ cells per well and incubated overnight. Unopsonized or IgG-opsonized beads were incubated with the cells at a concentration of 0.5×10⁷/ml for an hour. The cells were washed extensively in FACS buffer (PBS, 0.2% BSA, 0.01% sodium azide), briefly treated with trypsin, and evaluated by flow cytometry on a FACScan flow cytometer (Becton Dickinson, San Jose Calif.). Results were analyzed using CellQuest software. The phagocytic index (PI) was calculated from the following formula: phagocytic index=percent of cells containing beads X mean fluorescence of cells containing beads. The data shown in FIG. 3 represent the mean PI of at least six determinations for each cell line.

### Example 7

### Evaluation of Cell Surface Marker Expression

Expression of the cell surface markers murine F4/80, FcγRIA, FcγRIlB, FcγRIIIA, and FcR-γ was evaluated using both RT-PCR and flow cytometry.

Total RNA was isolated from mAM, mAM PU.1, and mAM PU.1+Evi1 cells. RT-PCR reactions were performed on the RNA. Primer sets specific to murine F4/80, FcγRIA, FcγRIIB, FcγRIIIA, and FcR-γ were used. All RT-PCR analyses were performed in duplicate in two separate experiments. Products of the RT-PCR were resolved on an agarose gel. Results from a typical experiment are presented in FIG. 3A.

AM, mAM PU.1, and mAM PU.1+Evi1 cells were cultured on plates. The cells were collected by scraping in Versene (Life Technologies, Grand Island N.Y.) then were resuspended in fluorescence activated cell sorter (FACS) buffer (PBS, 0.2% BSA, 0.01% sodium azide). The cells were washed with FACS buffer, counted, and divided into aliquots of 10⁵ cells in 100 μl FACS buffer. Cells were incubated with phycoerythrin-conjugated antibody directed at mouse FcγRII/III (Pharmingen) for 30 minutes at 4° C. As controls cells were also evaluated with primary isotype and species matched anti-mouse immunoglobulins. The immunostained cells were washed twice with FACS buffer, stored on ice, and analyzed by single-color flow cytometry using a FACScan flow cytometer as described elsewhere herein.

### Example 8

### Cellular Proliferation of Erythroid Leukemia Cells

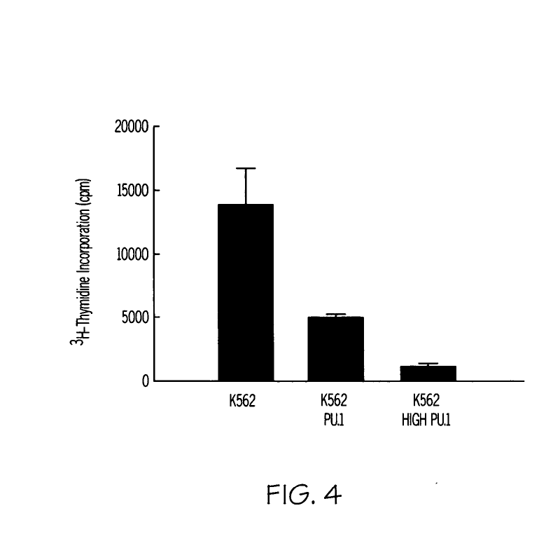

A modified murine stem cell virus (MSCV)-based bicistronic retroviral vector, MIEG3, expressing an enhanced green fluorescence protein (EGFP), was used to construct a MIEG3-PU.1-EGFP retroviral construct. K562 cells, an erythroid leukemic cell line, were maintained in IMEM medium supplemented with 10% fetal bovine serum, 2 mM/L L-glutamine, 10,000 u/ml penicillin, and 10 μg/ml streptomycin at 37° C. in a humidified atmosphere containing 5% CO₂, and passaged every 3 days. K562 cells were transfected with the MIEG3-PU.1-EGFP retroviral construct. Proliferation of K562, K562 PU.1, and K562 High PU.1 cells was assessed by determining the extent of DNA synthesis using a [³H]-thymidine incorporation assay. [³H]-thymidine incorporation assays are known in the art and described in Kurokawa, M et al. (1998) Nature 394:92-96, herein incorporated by reference in its entirety. Typical results are shown in FIG. 4.

### Example 9

### Lipopolysaccharide (LPS) Stimuli Response Assessment

AM, MS-H, MS-H Evi-1, mAM+PU.1, and mAM+PU.1+Evi-1 cells were plated at 2×10⁵ per 35 mm plate and incubated for one day. Then the cells were transformed with the ELAM-LUC plasmid (an NF-κB promoter-firefly luciferase reporter derived from reporter plasmid pGL3 (Promega)) and a renilla luciferase reporter control plasmid. Plasmids were transfected into cells using Effectine (QIAGEN, Valencia Calif.). The transfected cells were incubated for 24 hours. Cells were exposed to LPS (100 ng/ well) or a mock treatment. The cells were incubated an additional 24 hours. Firefly and renilla luciferase activities were quantified on a Monolight 3010 luminometer (Analytical Luminescence Laboratory, Ann Arbor Mich.) using the dual luciferase assay kit (Promega, Madison Wis.). Firefly luciferase values were normalized for renilla luciferase activity. Results from LPS treated cells were compared to results from untreated cells.

All publications, patents, and patent applications mentioned in the specification are indicative of the level of those skilled in the art to which this invention pertains. All publications, patents, and patent applications are herein incorporated by reference to the same extent as if each individual publication or patent application was specifically and individually incorporated by reference.

Having described the invention with reference to the exemplary embodiments, it is to be understood that it is not intended that any limitations or elements describing the exemplary embodiment set forth herein are to be incorporated into the meanings of the patent claims unless such limitations or elements are explicitly listed in the claims. Likewise, it is to be understood that it is not necessary to meet any or all of the identified advantages or objects of the invention disclose herein in order to fall within the scope of any claims, since the invention is defined by the claims and since inherent and/or unforeseen advantages of the present invention may exist even though they may not be explicitly discussed herein.

That which is claimed:

1. A myeloid cell stably transformed with an expression cassette comprising a promoter operably linked to a nucleotide sequence of interest having a nucleotide sequence selected from the group consisting of:
(a) a nucleotide sequence having the sequence set forth in SEQ ID NO:1;
(b) a nucleotide sequence having at least 90% identity to the sequence set forth in SEQ ID NO:1, wherein said sequence encodes a polypeptide having proliferating activity;
(c) a nucleotide sequence that encodes a polypeptide having the amino acid sequence set forth in SEQ ID NO:2; and
(d) a nucleotide sequence that encodes a polypeptide having at least 90% identity to the amino acid sequence set forth in SEQ ID NO:2, wherein said polypeptide has proliferating activity.

2. The myeloid cell of claim 1, wherein said myeloid cell is from a mammal.

3. The myeloid cell of claim 2, wherein said cell is selected from the group consisting of human, monkey, chimpanzee, mouse, rabbit, rat, hamster, dog, pig, goat, and cow.

4. The myeloid cell of claim 1, wherein said promoter is selected from the group consisting of constitutive and inducible promoters.

5. The myeloid cell of claim 1, further comprising a second expression cassette comprising a second promoter operably linked to a second nucleotide sequence of interest having a nucleotide sequence selected from the group consisting of:
(a) a nucleotide sequence having the sequence set forth in SEQ ID NO:3;
(b) a nucleotide sequence having at least 90% identity to the sequence set forth in SEQ ID NO:3, wherein said sequence encodes a polypeptide having differentiating activity;
(c) a nucleotide sequence that encodes a polypeptide having the amino acid sequence set forth in SEQ ID NO:4; and
(d) a nucleotide sequence that encodes a polypeptide having at least 90% identity to the amino acid sequence set forth in SEQ ID NO:4, wherein said polypeptide has differentiating activity.

6. A process of modulating differentiation of a myeloid cell, said process comprising the steps of:
(a) providing a myeloid cell of claim 1; and
(b) monitoring differentiation of said cell.

7. The process of claim 6 further comprising the step of transforming said cell with a second expression cassette comprising a second promoter operably linked to a second nucleotide sequence of interest selected from the group consisting of:
(a) a nucleotide sequence having the sequence set forth in SEQ ID NO:3;
(b) a nucleotide sequence having at least 90% identity to the sequence set forth in SEQ ID NO:3, wherein said sequence encodes a polypeptide having differentiating activity;
(c) a nucleotide sequence that encodes a polypeptide having the amino acid sequence set forth in SEQ ID NO:4; and
(d) a nucleotide sequence that encodes a polypeptide having at least 90% identity to the amino acid sequence set forth in SEQ ID NO:4, wherein said polypeptide has differentiating activity.

8. The process of claim 7, wherein said first and second expression cassettes are in one vector.

9. The process of claim 7, wherein said first and second expression cassettes are in multiple vectors.

10. A method for preparing a myeloid cell line suitable for tissue culture, said method comprising the steps of:
(a) providing a myeloid cell of claim 1; and
(b) selecting cells comprising the expression cassette.

11. The method of claim 10 further comprising the steps of:
(a) transforming said cells with a second expression cassette comprising a second promoter operably linked to a second nucleotide sequence of interest having a nucleotide sequence selected from the group consisting of:
(i) a nucleotide sequence having the sequence set forth in SEQ ID NO:3;
(ii) a nucleotide sequence having at least 90% identity to the sequence set forth in SEQ ID NO:3, wherein said sequence encodes a polypeptide having differentiating activity;
(iii) a nucleotide sequence that encodes a polypeptide having the amino acid sequence set forth in SEQ ID NO:4; and
(iv) a nucleotide sequence that encodes a polypeptide having at least 90% identity to the amino acid sequence set forth in SEQ ID NO:4, wherein said polypeptide has differentiating activity; and
(b) selecting cells comprising the second expression cassette.

12. The method of claim 10, further comprising the step of obtaining a clonal population.

13. A process of modulating differentiation of a myeloid cell line, said process comprising the steps of:
(a) providing a myeloid cell of claim 1; and
(b) monitoring differentiation of said cells.

14. A process of modulating differentiation of a myeloid cell line, said process comprising the steps of:
(a) providing a myeloid cell of claim 5; and
(b) monitoring differentiation of said cells.

15. A method for assaying the differentiating activity of a compound of interest, said method comprising the steps of:
(a) providing a myeloid cell of claim 1;
(b) administering said compound to said cell; and
(c) monitoring the differentiation of said cell.

16. The process of claim 15, wherein said cell differentiates into a cell type selected from the group consisting of macrophage, neutrophil, and mast cells.

17. A method for assaying the capability of a compound of interest to modulate a immune disorder comprising the steps of:
(a) providing a myeloid cell of claim 1;
(b) administering said compound to said cell; and
(c) monitoring an immune disorder associated phenotype of said cell.

18. The method of claim 17, wherein said phenotype is cellular maturity.

19. The method of claim 18, wherein cellular maturity increases.

20. The method of claim 17, wherein said immune disorder is a myeloid disorder.

21. The method of claim 20, wherein said myeloid disorder is selected from the group consisting of: acute myeloid leukemia, chronic myelogenous leukemia, and chronic granulomatous disease.

22. The method of claim 17, wherein said immune disorder is an erythroid disorder.

23. A method for assaying the capability of a compound of interest to treat an immune disorder comprising the steps of:
(a) providing a myeloid cell of claim 1;
(b) administering said compound to said cell; and
(c) monitoring an immune disorder associated phenotype of said cell.

24. The method of claim 23, wherein said immune disorder is a leukemia.

25. The method of claim 24, wherein said leukemia is selected from the group consisting of: acute myeloid leukemia, chronic myelogenous leukemia, and chronic granulomatous disease.

26. The method of claim 23, wherein said immune disorder is a myeloid disorder.

27. The method of claim 23, wherein said immune disorder is an erythroid disorder.

28. A method of identifying anti-leukemia agents comprising the steps of:
(a) providing a myeloid cell of claim 1;
(b) administering a compound of interest to said cell; and
(c) monitoring a leukemia associated phenotype.

29. A process of modulating Evi1 expression levels in a cell, said process comprising the steps of:
(a) isolating a myeloid cell; and
(b) transforming said cell with a first expression cassette comprising a first promoter operably linked to a nucleic acid molecule having a nucleotide sequence of interest selected from the group consisting of:
(i) a nucleotide sequence having the sequence set forth in SEQ ID NO:1;
(ii) a nucleotide sequence having at least 90% identity to the sequence set forth in SEQ ID NO:1, wherein said sequence encodes a polypeptide having proliferating activity;
(iii) a nucleotide sequence that encodes a polypeptide having the amino acid sequence set forth in SEQ ID NO:2; and
(iv) a nucleotide sequence that encodes a polypeptide having at least 90% identity to the amino acid sequence set forth in SEQ ID NO:2, wherein said polypeptide has proliferating activity.

30. The process of claim 29, comprising the step of isolating a myeloid cell.

31. The process of claim 29, further comprising the step of transforming said cell with a second expression cassette comprising a second promoter operably linked to a second nucleotide sequence of interest having a nucleotide sequence selected from the group consisting of:
(a) a nucleotide sequence having the sequence set forth in SEQ ID NO:3;
(b) a nucleotide sequence having at least 90% identity to the sequence set forth in SEQ ID NO:3, wherein said sequence encodes a polypeptide having differentiating activity;
(c) a nucleotide sequence that encodes a polypeptide having the amino acid sequence set forth in SEQ ID NO:4; and
(d) a nucleotide sequence that encodes a polypeptide having at least 90% identity to the amino acid sequence set forth in SEQ ID NO:4, wherein said polypeptide has differentiating activity.

32. A method of modulating the transition from the proliferation of undifferentiated macrophage lineage cells to differentiated macrophages, said method comprising the steps of:
(a) isolating macrophage lineage cells;
(b) transforming said cells with a first expression cassette comprising a first promoter operably linked to a first nucleic acid molecule having a nucleotide sequence selected from the group consisting of:
(i) a nucleotide sequence having the sequence set forth in SEQ ID NO:1;
(ii) a nucleotide sequence having at least 90% identity to the sequence set forth in SEQ ID NO:1, wherein said sequence encodes a polypeptide having proliferating activity;
(iii) a nucleotide sequence that encodes a polypeptide having the amino acid sequence set forth in SEQ ID NO:2; and
(iv) a nucleotide sequence that encodes a polypeptide having at least 90% identity to the amino acid sequence set forth in SEQ ID NO:2, wherein said polypeptide has proliferating activity; and
(c) monitoring differentiation of said cells.

33. The method of claim 32, wherein said proliferation is anchorage- independent.

34. The method of claim 32, wherein said proliferation is anchorage- dependent.

35. The method of claim 32, wherein said first promoter is selected from the group consisting of inducible and constitutive promoters.

36. The method of claim 32, further comprising the step of transforming said cells with a second expression cassette comprising a promoter operably linked to a nucleotide sequence of interest selected from the group consisting of:
(a) a nucleotide sequence having the sequence set forth in SEQ ID NO:3;
(b) a nucleotide sequence having at least 90% identity to the sequence set forth in SEQ ID NO:3, wherein said sequence encodes a polypeptide having differentiating activity;
(c) a nucleotide sequence that encodes a polypeptide having the amino acid sequence set forth in SEQ ID NO:4; and
(d) a nucleotide sequence that encodes a polypeptide having at least 90% identity to the amino acid sequence set forth in SEQ ID NO:4, wherein said polypeptide has differentiating activity.

37. The method of claim 32, wherein said differentiated macrophages have a reduced rate of proliferation.

38. The method of claim 32, wherein said differentiated macrophages are terminally differentiated.

39. The method of claim 32, wherein said differentiated macrophages are intermediately differentiated.
